(19) United States
(12) Patent Application Publication
(10) Pub. No.: US 2015/0273063 A1
(43) Pub. Date: Oct. 1, 2015

(54) MARKERS FOR PHARMACEUTICALS

(71) Applicants: Anthony KUHLMANN, San Diego,, CA (US); Ruprecht KELLER, Koln (DE)

(72) Inventor: Ruprecht Keller, Koln (DE)

(21) Appl. No.: 14/434,382

(22) PCT Filed: Sep. 18, 2013
(86) PCT No.: PCT/US13/60427

§ 371 Date: Apr. 8, 2015

Related U.S. Application Data
(60) Provisional application No. 61/712,099, filed on Oct. 10, 2012.
(60) Provisional application No. 61/840,253, filed on Jun. 27, 2013.

(51) Int. Cl. A61K 47/10; G01N 33/15

(52) U.S. Cl. CPC A61K 47/10; G01N 33/15

(57) ABSTRACT

Provided are methods for labeling a pharmaceutical product to indicate the origin, and/or intended recipient, and/or a predetermined characteristic (e.g. geographic location) of an intended recipient of the pharmaceutical product. The methods include incorporating certain pharmaceutically inactive marker substances into the pharmaceutical product at manufacture.

### CROSS-REFERENCE TO RELATED APPLICATIONS

This application claims benefit of U.S. Provisional Applications 61/712,099, filed Oct. 10, 2012, and 61/840,253, filed Jun. 27, 2013.

### TECHNICAL FIELD

The present invention is generally related to methods and compositions for marking pharmaceutical formulations for any purpose including, for example, to indicate the origin, and/or intended recipient, and/or a predetermined characteristic (e.g. geographic location) of an intended recipient of the pharmaceutical product.

### BACKGROUND OF THE INVENTION

The authentication and tracking of pharmaceutical products is becoming an increasing concern by governmental agencies, manufacturers, distributors, sellers, and consumers, for a variety of reasons. For example, there has been a recent proliferation of counterfeit medications (i.e., pharmaceuticals manufactured by an unauthorized manufacturer) which are made to appear similar or even identical to legitimate products and often sold on the so-called “black market” or integrated into the normal stream of commerce, being passed off as legitimate. Such counterfeit medications may be contaminated, contain the wrong or no active ingredient, or even have the correct active ingredient but at an incorrect dosage.

Manufacturers, distributors, and/or sellers have an interest in tracking the source of legitimate pharmaceutical products. As a result of regional or country specific regulatory, patent, or market conditions, some pharmaceuticals have significant price disparity between countries. In such instances, pharmaceutical products authorized or intended for sale by the manufacturer within one country (low-price) may find their way through the so-called “grey market” for sale in another (high-price) country at significantly reduced prices. These grey market sales of legitimate products result in the loss of revenue and profits available in that second country. Thus, there is a need to track legitimate pharmaceutical products to determine in which country or region that product was intended for sale, thereby combatting grey market traffic.

Tracking the source of pharmaceutical products is also useful in case of inadvertent contamination or other adulteration. Labeled pharmaceutical products may be tracked to determine the particular factory or even individual batch from which the contaminated or adulterated pharmaceutical product originated. This is particularly useful for dispensed products (e.g., prescription products) for which the original manufacturer's package may be unknown or unidentifiable at the time of contamination detection (i.e., when contamination is detected after the product has been dispensed to the consumer).

In all of these circumstances, it is desirable to have a means of authenticating or determining the source of the pharmaceutical product in question. In this context there is no question that any analytical investigation of a sample is only meaningful if the results obtained in the investigation can be used to determine if a pharmaceutical product was made by a particular manufacturer (in the case of authenticating potential black market goods), determine what geographic market was intended for the pharmaceutical product (in the case of authenticating potential grey market goods), or determine a particular factory or specific batch of origin (in the case of determining the origin of a pharmaceutical product), in order to then initiate the correct response.

### SUMMARY OF THE INVENTION

The present invention provides labeled pharmaceutical products, and methods for making the same, which may be used to indicate or trace any aspect of the pharmaceutical product including, for example, its site of manufacture, intended recipient (e.g., geographical or other market for intended sale), and as a marker of authenticity.

In one aspect, the invention provides a method of labeling a pharmaceutical product, said method comprising incorporating a unique marker profile in the pharmaceutical product during manufacture, wherein said unique marker profile comprises one or more pharmaceutically inactive marker substances. The marker substance(s) may be incorporated into the pharmaceutical product at any point during the manufacturing process according to known techniques appropriate for the particular formulation (e.g., tablet, oral solution or suspension, injectable, etc.) and marker substance.

In another aspect, the invention provides a labeled pharmaceutical product comprising:
a pharmaceutically active ingredient; and
a unique marker profile encoding one or more of the origin of the pharmaceutical product, the intended recipient of the pharmaceutical product, and a preselected characteristic of an intended recipient of the pharmaceutical product;
wherein said unique marker profile comprises one or more pharmaceutically inactive marker substances, and wherein said pharmaceutically inactive marker substances are not identifiable by sequencing.

In some embodiments of any of the aspects of the invention, the pharmaceutical product contains at least two, three, four, five, or more marker substances.

Suitable marker substances include, for example, carbohydrates, disaccharides, trisaccharides, tetrasaccharides, oligosaccharides, polysaccharides, isoprenoids, lipids, steroids, polyethylene glycols (monodispersed and/or polydispersed), acrylic polymers, poloxamers, polyoxyls, polysorbates, acesulfame, an acetylated monoglyceride, butylparaben, povidone, copovidone, crospovidone, gelucire, hypromelloses, polycarbophil, polydextrose, tartaric acid or a salt thereof, and derivatives thereof. When polymers (e.g., polyethylene glycols) are used as marker substances, polymers of different molecular weights may be used as individual (i.e., different) marker substances. In some embodiments, none of the marker substances are identifiable by sequencing (e.g., none of the marker substances comprise a nucleic acid).

In some embodiments, marker substances are substances identifiable or quantifiable by enzymatic, immunological, spectrometric, or electrophoretic methods and/or by an instrumental analytical chemistry technique including, for example, mass spectrometry (e.g., Gas Chromatography/Mass Spectrometry (GC/MS), Gas Chromatography/Tandem Mass Spectrometry (GC/MS/MS), High Performance Liquid Chromatography/Mass Spectrometry (HPLC/MS), High Performance Liquid Chromatography/Tandem Mass Spectrometry (HPLC/MS/MS), High Performance Liquid Chromatography (HPLC), or Gas Chromatography (GC)).

In some embodiments, the unique marker profile comprises two or more pharmaceutically inactive marker substances, and at least two of said pharmaceutically inactive marker substances are the same type of marker substances. Alternatively, at least two of said pharmaceutically inactive marker substances are different types of marker substances.

In certain preferred embodiments, the marker substances include a disaccharide, trisaccharide, tetrasaccharide, oligosaccharide, or polysaccharide. In other preferred embodiments, the marker substances include a polydisperse polyethylene glycol and/or a monodisperse polyethylene glycol. Particularly useful marker profiles comprise two or more monodisperse polyethylene glycols each with different molecular weights.

In some embodiments, the unique marker profile indicates the origin of said pharmaceutical product (e.g., the country, region, or manufacturing site (production facility)), the intended recipient of said pharmaceutical product (e.g., patient population) and/or a preselected characteristic of an intended recipient of said pharmaceutical product (e.g., geographic region in which an intended recipient is located). Some of the embodiments described above recite a preselected characteristic of an intended recipient of a pharmaceutical product. This characteristic may be any characteristic useful to group or categorize intended recipients. For example, the unique marker profile may include marker substances which indicate an intended recipient is found within a particular geographic region. In another example, the unique marker profile may include marker substances which indicate an intended recipient is subject to review by a particular regulatory or other governmental agency.

In some embodiments, the identification of the marker profile and its indication may be based on the identity of the marker substances alone (i.e., the presence or absence of the marker substances), the relative amounts of the marker substances (i.e., the ratios of the marker substances relative to each other in the pharmaceutical product), the absolute amounts of the marker substances in the pharmaceutical product, or some combination thereof.

As used herein, the term “origin of a pharmaceutical product” is an absolute term denoting the location or facility where a pharmaceutical product was manufactured.

As used herein, the term “source of a pharmaceutical product” is a relative term denoting the supplier of a pharmaceutical product. In some instances, the source of a pharmaceutical product may be the same as the place of origin, i.e., the location or facility of origin. In other instances, pharmaceutical products may be shipped from their location or facility of origin to a recipient, who may then themselves become a source of the pharmaceutical product if they provide the pharmaceutical products to another party.

As used herein, the term “derivative” is to be understood as all subsequent products which arise as a result of an induced or naturally occurring chemical transformation of a substance. Derivatives of marker substances can arise in the organism of the subject (e.g., by metabolism of the marker substance), or in a sample by induced or naturally occurring chemical transformation.

As used herein, the term “quantity” is to be understood as the amount of a particular substance and may be used to describe either an absolute measure of a substance in a sample, or to describe the relative amount of a substance in a sample with respect to some other substance(s) in the sample. Likewise, the term “quantify” is to be understood as to determine an absolute measure of a substance in a sample, or determine the relative amount of a substance in a sample with respect to some other substance(s) in the sample.

### DETAILED DESCRIPTION

The following description and examples describe in detail exemplary embodiments of pharmaceutical compositions which allow for authentication and/or determination of source, and methods for using the same. It should be appreciated that there are numerous variations and modifications of the compositions and methods described herein that are encompassed by the present invention. Accordingly, the description of a certain exemplary embodiment should not be deemed to limit the scope of the present invention.

In one aspect, the invention provides a pharmaceutical composition comprising one or more marker substances which are added during the manufacturing process, i.e., at the place of origin. These markers can be employed in a variety of embodiments, several of which are described below, e.g., to authenticate a particular factory of manufacture, or determine the intended country or regional market for which a particular pharmaceutical product was intended.

### Marker Substances

Any suitable marker substance or combination of marker substances may be used as described herein. A marker substance or combination of marker substances includes any substance or combination of substances that can be added to the intended pharmaceutical product without affecting the pharmacological behavior or usefulness of the pharmaceutical product, while providing a unique label associated with a particular source or intended recipient.

Advantageous marker substances are characterized in that they are detectable by known and routine detection methods already established in chemical investigation laboratories, such as for example common methods of clinical analytical chemistry. Such marker substances may or may not be taken up by the body upon administration, and are preferably FDA recognized as acceptable inactive ingredients for pharmaceutical formulations.

Exemplary marker substances may be drawn from a number of different types of chemical species, such as:

Carbohydrates, such as heptuloses, hexoses, pentoses, tetroses, trioses, in natural, oxidized, or reduced forms;

Disaccharides, such as lactose and chitobiose;

Tri-, tetra-, or oligosaccharides, such as N- or O-linked glycoprotein oligosaccharides,

Polysaccharides, such as starches, mannane, xylene, cellulose, hemicelluloses, and cleavage products or derivatives thereof;

Isoprenoids, such as dolichole or dolichole phosphate,

Lipids, such as triglycerides, stearines, and other fatty acids,

Steroids, such as cholesterol and its derivatives;

Polyethylene glycols (including monodisperse and polydisperse polyethylene glycols);

Acrylic polymers, such as carbomers and carboxypolymethylenes;

Poloxamers;

Polyoxyls;

Polysorbates;

polyethylene glycol (PEG)

acesulfame and salts thereof (e.g., acesulfame potassium)

acetylated monoglycerides

butylparaben (butyl parahydroxybenzoate)

povidone (polyvinylpyrrolidone)

copovidone (crospovidone)

gelucires (mixture of glycerides and esters of polyethylene glycol including, for example, mono-, di- and triglycerides and mono- and diesters of PEG; e.g., gelucire 33/01, 37/02, 39/01, 43/01, 44/14, 50/02, 50/13, 53/10, and 62/02)

hypromelloses (hydroxypropyl methyl cellulose)

polycarbophil (polyacrylic acid cross-linked with divinyl glycol)

polydextrose,

tartaric acid and salts thereof (2,3-dihydroxybutanedioic acid)

and derivatives or mixtures of these substances.

In some embodiments, the marker substances are not natural or synthetic polypeptides, nucleic acids, or other non-nucleic acid polymers that are identifiable by sequencing.

When using polymers as marker substances (e.g., PEG, including glucires, celluloses, polyacrylic acids, polydextrose, etc.), the molecular weight of the polymers is preferably less than about 5000 Da, 4000 Da, 3000 Da, 2000 Da, 1,500 Da, 1000 Da, 900 Da, 800 Da, 700 Da, 600 Da, 500 Da, or 400 Da, and/or greater than about 100 Da, 150 Da, 200 Da, 250 Da, 300 Da, 400 Da, and 500 Da. Typically, the polymer has at least about 2, 3, 4, 5, 7, 10, 15, 20, 25, 30, 40, 50, 75, or 100 repeating monomeric units and/or not more than about 15, 25, 50, 75, 100, 150, 200, 250, or 500 repeating monomeric units.

In embodiments where the marker substances include one or more sugars, one or more sugars may be selected from the group consisting of arabinose, erythrulose, myo-inositol, cis-inositol, mannitol, sorbose, rhamnose, sorbitol, xylose and xylulose, or any other sugar which is soluble in water. Preferably, when sugars or their derivatives are used as marker substances, the marker substances can be easily detected by routine, known chemical evaluation techniques, such as enzymatic tests.

In embodiments where the marker substances include two or more polyethylene glycols, each polyethylene glycol should be distinguishable by chemical and/or physical analysis. For example, each polyethylene glycol in the marker substance should have a different molecular weight. In this way, the identity and/or quantity and/or determine the relative amounts of each polyethylene glycol in the combination of marker substances may be determined e.g., by various forms of mass spectrometry.

In some embodiments, the marker substances are substances which are not absorbed by a subject who has taken the pharmaceutical. In these embodiments, determination of the unique pharmaceutical marker may typically be conducted by analyzing a sample of the pharmaceutical product itself

In other embodiments, the marker substances are substances which are absorbed by a subject who has taken the pharmaceutical, and which can be detected in one or more body fluids, such as urine, blood, plasma, serum or the like, of the subject. In some related embodiments, the marker substances are not metabolized following uptake by the subject. In other related embodiments, the marker substances are metabolized to a derivative specifically attributable to the marker substance. In embodiments where the marker substances are absorbed by a subject who has taken the pharmaceutical and are either not metabolized or metabolized to a derivative specifically attributable to the marker substances, determination of the unique marker profile may be conducted by analyzing a sample of the pharmaceutical product itself, as above, or by analyzing a biological sample obtained from a person who has consumed or otherwise been administered the pharmaceutical product.

In some embodiments, if the marker substances are soluble in a liquid based pharmaceutical product, the normal taste of the liquid is not changed by their addition.

Incorporation of the Marker Substance(s) into a Pharmaceutical Product

It is preferable to use a marker substance or combination of multiple marker substances to label a particular pharmaceutical product in a way that allows for determination of origin and/or source with a relatively high degree of certainty. In some instances, this may involve adding a single marker substance to the pharmaceutical during manufacture at a non-routine concentration, or adding combinations of 2, 3, 4, 5, or more marker substances, each at its own independent concentration. It is readily apparent that the more marker substances that are present, each at their own independent levels of concentration, the more unlikely a particular combination is to be used as excipient in the routine manufacture of a pharmaceutical product.

As used herein, the term “pharmaceutical product” is intended to generally encompass all delivery vehicles for administration of an active to a patient. The concept is not to be limited to route of administration. Exemplary solid form pharmaceutical products include tablets, capsuls, gelcaps, powders, granules, and the like. Exemplary liquid form pharmaceutical products include forms suitable for ocular, nasal, topical, or oral administration, and injectable formulations including those intended for administration by the intravenous, intramuscular, intrathecal, and subcutaneous routes.

When incorporated into solid-form pharmaceutical products, the maker substances may be incorporated into any component of the product. For example, the markers substances may be included in the main formulation as described above, and added as inactive ingredient(s). Alternatively, the marker substances may be included as part of any other component of the formulation, such as a coating or gel cap, if such is used in the product.

As described above, a number of different marker substances may be used in various embodiments of the invention. However, not all marker substances may be suitable for all possible pharmaceutical formulation forms. For example, marker substances suitable for inclusion in a tablet or capsule may be different than those that are suitable for inclusion in a liquid formulation. It is apparent to one of skill in the art which marker substances are suitable for particular pharmaceutical forms.

Once suitable marker substances have been selected, the marker substances are added to a formulation at the manufacturing site. These additions are typically included as inactive ingredients comprising a percentage (by weight) of the total formulation. The mechanics of these additions will vary according to the particular pharmaceutical formulation being labeled and the nature and amount of the marker substances being included, but will be readily determined by those of skill in the art.

Methods of Identifying the Source and/or Intended Recipients of a Pharmaceutical Product

The number of marker substances and amount of each included in a pharmaceutical product may be varied to achieve any of the uses described herein. For example, each of plurality of manufacturing facilities may be assigned a unique marker profile (i.e., a unique set of marker substances, optionally including the absolute or relative concentrations of each marker substance) that is incorporated into pharmaceutical products manufactured at that facility. In this way, the particular manufacturing plant of any individual pharmaceutical product (i.e., a pharmaceutical product's place of origin) may later be identified by determining the identities (and possibly amounts) of marker substances contained in the pharmaceutical product and comparing this information to known marker profiles for each possible manufacturing facility. Such a determination facilitates authentication of a pharmaceutical product, and thus aids in identification of counterfeit pharmaceuticals.

In another embodiment, a larger number of unique marker profiles may be generated from a smaller number of marker substances by varying the relative concentrations of the marker substances in known/pre-determined ratios. For example, in its simplest form when only two marker substances are used, represented generically as X and X′, multiple marker profiles may be constructed by varying the ratios of X to X′ as follows:

Relative Amount of Marker Substance

| Marker # | X | X′ |
|---|---|---|
| 1 | 1 | 0 |
| 2 | 0 | 1 |
| 3 | 1 | 1 |
| 4 | 1 | 2 |
| 5 | 1 | 3 |
| 6 | 1 | 4 |
| 7 | 2 | 1 |
| 8 | 3 | 1 |
| 9 | 4 | 1 |

The ratios of the paired marker substances are not limited to those ratios shown in the table above but may include any convenient ratio or combination of ratios such as 5:1, 10:1, 15:1, 20:1, or more. The only practical limits are those of convenience and detectability. Furthermore, the strategy of constructing unique marker profiles based on the relative ratios of marker substances is not limited to pairs of marker substances but instead can be extended to varying the relative ratios of 3, 4, 5, 6, 7, 8, or more marker substances, thereby significantly increasing the total number of unique marker profiles available for any given number of marker substances, without increasing the number of chemically distinct marker substances.

In some embodiments, every intended recipient of a pharmaceutical product may be assigned a unique marker profile (i.e., a unique set of marker substances, optionally including the absolute or relative concentrations of each marker substance) that is incorporated into the formulation of the pharmaceutical product during manufacture. Later identification of this unique marker profile in a particular pharmaceutical product allows for determination of the intended recipient of the pharmaceutical product. Such a determination facilitates identification and investigation of black market and grey market pharmaceuticals.

In some embodiments, all intended recipients meeting a particular criterion (e.g., those within a certain geographic region, or those subject to a common set of laws and/or regulations) are assigned a unique marker profile (i.e., a unique set of marker substances, optionally including the absolute or relative concentrations of each marker substance) that is incorporated into the formulation of the pharmaceutical product during manufacture. Thus, pharmaceutical products made for all intended recipients meeting a particular criterion include a unique marker. Later identification of this unique marker profile in a particular pharmaceutical product allows for determination of the group of intended recipients that share a preselected criterion. Such a determination facilitates identification and investigation of black market and grey market pharmaceuticals.

It should be understood that in some embodiments, the identity of a marker substance and the quantity of that marker substance may each be used to convey different information. The following example is not intended to be limiting and is intended to illustrate one possible embodiment. Take for example a scenario where some particular pharmaceutical is prepared in capsule form by a Manufacturing Facility 1 with two different Intended Recipients. Manufacturing Facility 1 may be assigned a unique marker profile comprising two marker substances, A and B. Thus, all capsules manufactured at Manufacturing Facility 1 include both marker substances A and B. However, the amounts (absolute or relative) of the two marker substances may be varied to indicate the intended recipient. For example, capsules manufactured for Intended Recipient 1 may comprise some amount of marker substance A, and twice that amount of marker substance B. Capsules manufactured for Intended Recipient 2 may comprise some amount of marker substance A, and one half that amount of marker substance B. In this way, later analysis of the identity of the marker substances allows for determination of the source of the pharmaceutical (i.e., Manufacturing Facility 1 because of the presence of both marker substances A and B), and analysis of the relative amounts of the marker substances allows for the determination of the intended recipient of the capsule.

In other embodiments, each piece of information that is to be conveyed by the marker substances is encoded with at least one different marker substance. The following illustrative example is provided to contrast the example described above. Consider the same scenario as above where some particular pharmaceutical is prepared in capsule form by Manufacturing Facility 1 with two different Intended Recipients. Manufacturing Facility 1 may be assigned a unique marker profile comprising two marker substances, A and B. Thus, all capsules manufactured at Manufacturing Facility 1 include both marker substances A and B. However, in these embodiments, one or more additional marker substances are used to encode the identity of the intended recipients. For example, capsules manufactured for Intended Recipient 1 may comprise some amount of an additional marker substance C, while capsules manufactured for Intended Recipient 2 may comprise some amount of additional marker substances D and E. In this way, later analysis of the identity of the marker substances in a capsules from this facility would show the presence of marker substances A and B, and either C or D and E. The identification of marker substances A and B allows for determination of the source of the pharmaceutical (i.e., Manufacturing Facility 1), and identification of marker substances C or D and E allows for the determination of the intended recipient of the pharmaceutical.

The above illustrative examples demonstrate that in some embodiments a plurality of marker substances may be used. In fact, the above examples may be considered to be relatively simple, in as far as the number of manufacturing facilities and intended recipients in the examples are very limited. However, it should be appreciated that the number of marker substances that can be used in any given unique marker profile is only limited by the number of suitable marker substances available. It should also be appreciated that a unique marker profile may contain as few as a single marker substance, with or without consideration of its relative or absolute concentration in the pharmaceutical product.

It is especially preferable to include multiple marker substances in a pharmaceutical product, wherein it is possible by the combination of marker substances to develop a certain numerical code belonging to a respective sample. For example, it is preferred to include a combination of at least 2, such as at least 3, such as at least 4, such as at least 5 marker substances simultaneously. Using a total of n marker substances, there exist 2ⁿ⁻¹ different combinations in a dual numeric system; that is without use of absolute or relative concentrations of each marker as additional indicia.

In some embodiments, a unique marker profile comprises a plurality of marker substances, and two or more of the plurality of marker substances are of the same type of marker substances (e.g. two or more sugars, two or more isoprenoids, two or more lipids, two or more saccharides, two or more starches, two or more polyols, two or more polyethylene glycols, etc. as described elsewhere). In some related embodiments, every marker substance in the plurality of marker substances are of the same type of marker substance.

In some embodiments a unique marker profile comprises a plurality of marker substances, and two or more of the plurality of marker substances are different types of marker substances (e.g. one sugar and one lipid, one isoprenoid and one starch, one polyol and one polyethylene glycols, etc. as described elsewhere). In some related embodiments, no two marker substances in the plurality of marker substances are of the same type of marker substance.

In some embodiments a unique marker profile comprises three or more marker substances, and two or more marker substances are of the same type of marker substance, while at least one of the three or more is of a different type of marker substance.

As described above, in some embodiments, at least one marker substance may not be absorbed by the body upon administration. In these embodiments, determination of the marker profile may be conducted by analyzing the pharmaceutical product itself. For example, if the pharmaceutical product is a capsule, at least a portion of the capsules may be crushed, dissolved in an appropriate solvent, and an aliquot of the resulting solution analyzed by the appropriate technique to identify and/or quantitate and/or determine the relative amounts of each marker substance present in the capsule. Once the unique marker profile has been identified, this information can be used to determine the site of origin and/or intended recipient or recipients of the capsule.

Also as described above, one or more marker substances may be substances which are absorbed by a subject who has taken the pharmaceutical product, and which can be detected in one or more body fluids, such as urine, blood, plasma, serum or the like, of the subject. In these embodiments, determination of the unique marker profile may be conducted by analyzing a sample of the pharmaceutical product itself, as above, or by analyzing a biological sample obtained from a person who has consumed or otherwise been administered the pharmaceutical product.

For example, consider a pharmaceutical liquid composition, such as a prescription cough syrup, that may be find use as an illicit drug. If a patient is confirmed to have illegally obtained and used the pharmaceutical liquid composition, an appropriate body fluid from the patient, such as a urine or blood sample, can be analyzed to identify the unique marker profile of the consumed cough syrup, so that it can be determined if the patient obtained the liquid composition from black market, grey market, or otherwise legitimate sources. This information may potentially assist law enforcement in tracking the illicit supplier of the pharmaceutical liquid composition.

In embodiments where a body fluid from a patient is to be analyzed for detection of the marker substances, any means of sample collection known to be suitable in the art can be used. For example, sample removal occurs in different ways depending on the type of sample to be investigated. In the case of the analysis of body excretions, part of the sample is taken up into a sample vessel and, after this time, is ready for further investigation. In the investigation of human urine or stool samples, the samples can usually be furnished by the subjects themselves in that the subject is simply given a sample vessel. For the removal of samples from body fluids or from tissue samples, a direct operation on the subject is normally necessary. Here, obtention of blood from the subject can be accomplished using a suction pipette following pricking or cutting of the skin with a disposable lancet or—in larger quantities—using an injection syringe or blood collection tube after puncture of the vein. For the investigation of liquor, the latter is obtained by lumbar, suboccipital or ventricle puncture.

By “biological sample” it is meant the components of a mammal designated for the analytical investigation. Relevant here are body excretions, body fluids or tissue samples. The components making up the sample can include components of a mammalian organism which still exist in the mammal at the time of sample removal as well as previous components of the mammal. By “body excretions” or “excretion” are to be understood urine, stool, secretions from salivary, milk, tear and sweat glands.

The term “body fluid” is to be understood as extracellular liquids of a mammalian organism (including male or female humans) like blood, serum and liquor. Preferably, the samples removed from or excreted by a mammal are body excretions, body fluids or tissue samples.

The term “tissue sample” is to be understood as an organization of identically differentiated cells obtained by a direct operation into the living mammalian organism, as well as these cells' intercellular substance. Hair samples and samples of sloughed-off parts of skin are also to be understood as falling within the meaning of this term.

Depending on the type of the sample and the at least one marker substance to be detected, the respective sample may have to be prepared prior to the analysis method. The preparation steps can include centrifugation for the separation of solid, non-solubilized materials in a liquid sample, solubilization or suspension of solid samples, concentration, precipitation with suitable reagents such as ammonium sulfate, adjustment of the pH value required for the analysis method, homogenization of the sample such as by ultrasonication or by using vibration cell mills, separation of materials used in lysing the sample such as for example detergents and other preparation steps known to one of ordinary skill in the art.

A number of enzymatic, immunological, mass spectrometric and electrophoretic detection methods as well as combinations of these methods are available for determining the identity and/or quantity and/or relative amounts of at least one marker substance in a sample. Preferably, analysis is conducted by an instrumental analytical chemistry technique, such as coupled Gas Chromatography/Mass Spectrometry (GC/MS) (with single or tandem MS), High Performance Liquid Chromatography/Mass Spectrometry (HPLC/MS) (with single or tandem MS), High Performance Liquid Chromatography (HPLC), or Gas Chromatography (GC). These methods allow the very time-efficient investigation of, in particular, liquid samples or of samples which, due to their preparation were transferred into a liquid. At the same time, these detection methods allow a high degree of automation so that a multitude of samples can be analyzed in a short time and, in as far the chromatograms and, as the case may be, mass spectroscopic fractionation patterns of reference substances already exist in the computer evaluation unit, the analysis of the at least one marker substance is also greatly simplified.

### Example 1

As an example, an embodiment for uniquely marking of a liquid pharmaceutical product to enable later identification of the manufacturing facility of origin is provided below.

A particular liquid pharmaceutical product is manufactured at three manufacturing facilities: Facility 1, Facility 2, and Facility 3. Each manufacturing facility is assigned a unique marker profile comprising two marker substances. These two marker substances are added in equal amounts during the manufacture of the liquid pharmaceutical, such that the total concentration of the marker substances is about 1% of the liquid pharmaceutical.

At Facility 1, the liquid pharmaceutical product is produced such that the product comprises about 0.5% wt of Marker A, a monodisperse polyethylene glycol with an approximate molecular weight of about 530 amu, and about 0.5% wt of Marker B, a monodisperse polyethylene glycol with an approximate molecular weight of about 574 amu.

At Facility 2, the liquid pharmaceutical product is produced such that the product comprises about 0.5% wt of Marker A, and about 0.5% wt of Marker C, a monodisperse polyethylene glycol with an approximate molecular weight of about 618 amu.

At Facility 3, the liquid pharmaceutical product is produced such that the product comprises about 0.5% wt of Marker B, and about 0.5% wt of Marker C.

After production, the products are distributed to wholesale suppliers, and ultimately to approved retail locations, where the products are available for purchase by patients with an appropriate prescription. If a liquid pharmaceutical product purported to be genuine is later found at an unapproved retail location, the responsible law enforcement or regulatory body may send a sample of the purportedly genuine liquid pharmaceutical product for mass spectrometric analysis for identification and quantitation of marker substances present in the product.

Several possible results of such analysis, and their meanings with respect to origin are presented below in Table 1.

TABLE 1

| Possible Outcome of Mass Spectrometric | |
|---|---|
| Analysis | Origin |
| Markers A and B, at about 1:1 | Facility 1 |
| Markers A and C, at about 1:1 | Facility 2 |
| Markers B and C, at about 1:1 | Facility 3 |
| No Markers present | Unknown (counterfeit) |
| Single Marker present | Unknown (counterfeit) |
| Two Markers present, but not at about 1:1 | Unknown (counterfeit) |
| Three or more Markers present | Unknown (counterfeit) |

### Example 2

As an example, an embodiment for uniquely marking of a liquid pharmaceutical product to enable later identification of the manufacturing facility of origin, and intended recipient is provided below.

A particular liquid pharmaceutical product is manufactured at three manufacturing facilities: Facility 1 and Facility 2. There are two intended recipients for the pharmaceutical products, and each intended recipient may receive pharmaceutical products from either Facility. The intended recipients for the product are Intended Recipient 1 and Intended Recipient 2.

Each combination of manufacturing facility and intended recipient is assigned a unique marker profile comprising four marker substances. Two marker substances are added in equal amounts during the manufacture of the liquid pharmaceutical, such that the total concentration of these two marker substances is about 1% of the liquid pharmaceutical. Two other marker substances are added at a ratio of 2:1 during the manufacture of the liquid pharmaceutical, such that the total concentration of these marker substances is about 1.5% of the liquid pharmaceutical. Thus, the total concentration of the four marker substances is about 2.5% of the total liquid pharmaceutical.

At Facility 1, all liquid pharmaceutical products, regardless of intended recipient, are produced such that the product comprises about 0.5% wt of Marker A, a monodisperse polyethylene glycol with an approximate molecular weight of about 530 amu, and about 0.5% wt of Marker B, a monodisperse polyethylene glycol with an approximate molecular weight of about 574 amu.

At Facility 2, all liquid pharmaceutical products, regardless of intended recipient, are produced such that the product comprises about 0.5% wt of Marker A, and about 0.5% wt of Marker C, a monodisperse polyethylene glycol with an approximate molecular weight of about 618 amu.

All liquid pharmaceutical products intended for Intended Recipient 1, regardless of production facility, are produced such that the product comprises about 0.5% wt of Marker D, a monodisperse polyethylene glycol with an approximate molecular weight of about 662 amu, and about 1% wt of Marker E, a monodisperse polyethylene glycol with an approximate molecular weight of about 706 amu.

All liquid pharmaceutical products intended for Intended Recipient 1, regardless of production facility, are produced such that the product comprises about 1% wt of Marker D, and about 0.5% wt of Marker E.

After production, the products are distributed to wholesale suppliers, and ultimately to approved retail locations, where the products are available for purchase by patients with an appropriate prescription. If a liquid pharmaceutical product purported to be genuine is later found at an unapproved retail location, the responsible law enforcement or regulatory body may send a sample of the purportedly genuine liquid pharmaceutical product for mass spectrometric analysis.

Several possible results of such analysis, and their meanings with respect to origin and intended recipient are presented below in Table 2.

TABLE 2

| Possible Outcome of Mass Spectrometric | | Intended |
|---|---|---|
| Analysis | Origin | Recipient |
| Markers A, B, D, E; present at about 1:1:1:2 | Facility 1 | Intended |
| | | Recipient 1 |
| Markers A, B, D, E; present at about 1:1:2:1 | Facility 1 | Intended |
| | | Recipient 2 |
| Markers A, C, D, E; present at about 1:1:1:2 | Facility 2 | Intended |
| | | Recipient 1 |
| Markers A, C, D, E; present at about 1:1:2:1 | Facility 2 | Intended |
| | | Recipient 2 |
| No Markers present | Unknown | N/A |
| | (counterfeit) | |
| Single Marker present | Unknown | N/A |
| | (counterfeit) | |
| Two Markers present | Unknown | N/A |
| | (counterfeit) | |
| Three Markers present | Unknown | N/A |
| | (counterfeit) | |
| Four Markers present, but wrong | Unknown | N/A |
| combination | (counterfeit) | |
| Four Markers present, but at incorrect ratio | Unknown | N/A |
| | (counterfeit) | |
| Five or more Markers present | Unknown | N/A |
| | (counterfeit) | |

If the pharmaceutical product under investigation is found to lack Markers A and B, or A and C, then the pharmaceutical product is known to be counterfeit.

If the pharmaceutical product is validated as genuine as to source but is found on the black or grey market, knowledge of the intended recipient, and thus the intended distribution network through which that product was intended to proceed, may prove useful in investigating the break in the supply chain which lead to availability of the pharmaceutical product on the black or grey market.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

Terms and phrases used in this document, and variations thereof, unless otherwise expressly stated, should be construed as open ended as opposed to limiting. As examples of the foregoing, the term ‘including’ should be read to mean ‘including, without limitation’ or the like; the term ‘comprising’ as used herein is synonymous with ‘including,’ ‘containing,’ or ‘characterized by,’ and is inclusive or open-ended and does not exclude additional, unrecited elements or method steps; the term ‘example’ is used to provide exemplary instances of the item in discussion, not an exhaustive or limiting list thereof; and adjectives such as ‘known,’ ‘normal,’ ‘standard,’ and terms of similar meaning should not be construed as limiting the item described to a given time period or to an item available as of a given time, but instead should be read to encompass known, normal, or standard technologies that may be available or known now or at any time in the future. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise. In addition, as used in this application, the articles ‘a’ and ‘an’ should be construed as referring to one or more than one (i.e., to at least one) of the grammatical objects of the article. By way of example, ‘an element’ means one element or more than one element.

The presence in some instances of broadening words and phrases such as ‘one or more,’ ‘at least,’ ‘but not limited to,’ or other like phrases shall not be read to mean that the narrower case is intended or required in instances where such broadening phrases may be absent.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1.-33. (canceled)

34. A method of labeling a pharmaceutical product, said method comprising incorporating into the pharmaceutical product during manufacture a unique marker profile comprising pharmaceutically inactive marker substances wherein said marker substances comprises two or more polyethylene glycols having different molecular weights.

35. The method of claim 34, wherein said unique marker profile further comprises one or more additional pharmaceutically inactive marker substances.

36. The method of claim 34, wherein said unique marker profile further comprises five or more additional pharmaceutically inactive marker substances.

37. The method of claim 35, wherein at least one of said additional pharmaceutically inactive marker substances is selected from the group consisting of carbohydrates, disaccharides, trisaccharides, tetrasaccharides, oligosaccharides, polysaccharides, isoprenoids, lipids, steroids, acrylic polymers, poloxamers, polyoxyls, polysorbates, acesulfame, an acetylated monoglyceride, butylparaben, povidone, copovidone, crospovidone, gelucire, hypromelloses, polycarbophil, polydextrose, tartaric acid or a salt thereof, and derivatives thereof.

38. The method of claim 34, wherein said unique marker profile comprises at least one monodisperse polyethylene glycol.

39. The method of claim 34, wherein said unique marker profile comprises two or more monodisperse polyethylene glycols each having a different molecular weights.

40. The method of claim 34, wherein said unique marker profile comprises one or more marker substances whose identities indicate the origin of said pharmaceutical product.

41. The method of claim 34, wherein said unique marker profile comprises two or more marker substances whose identities and absolute or relative amounts indicate the origin of said pharmaceutical product.

42. The method of claim 34, wherein said unique marker profile comprises one or more marker substances whose identities indicate the intended recipient of said pharmaceutical product.

43. The method of claim 34, wherein said unique marker profile comprises two or more marker substances, whose identities and absolute or relative amounts indicate the intended recipient of said pharmaceutical product.

44. The method of claim 34, wherein said unique marker profile comprises one or more marker substances, whose identities indicate a preselected characteristic of an intended recipient of said pharmaceutical product.

45. The method of claim 44, wherein said preselected characteristic is a geographic region in which an intended recipient is located.

46. The method of claim 34, wherein said unique marker profile comprises two or more marker substances whose identities and absolute or relative amounts indicate a preselected characteristic of an intended recipient of said pharmaceutical product.

47. The method of claim 46, wherein said preselected characteristic is a geographic region in which the intended recipient is located.

48. The method of claim 34, wherein said unique marker profile comprises two or more marker substances whose identities indicate two or more selected from the group consisting of the origin of the pharmaceutical product, the intended recipient of the pharmaceutical product, and a preselected characteristic of an intended recipient of the pharmaceutical product.

49. The method of claim 34, wherein said unique marker profile comprises two or more marker substances whose identities and absolute or relative amounts indicate two or more selected from the group consisting of the origin of the pharmaceutical product, the intended recipient of the pharmaceutical product, and a preselected characteristic of an intended recipient of the pharmaceutical product.

50. A labeled pharmaceutical product comprising:
a pharmaceutically active ingredient; and
a unique marker profile encoding one or more of the origin of the pharmaceutical product, the intended recipient of the pharmaceutical product, and a preselected characteristic of an intended recipient of the pharmaceutical product;
wherein said unique marker profile comprises pharmaceutically inactive marker substances wherein said marker substances comprises two or more polyethylene glycols having different molecular weights.

51. The labeled pharmaceutical product of claim 50, wherein said unique marker profile further comprises one or more additional pharmaceutically inactive marker substances.

52. The labeled pharmaceutical product of claim 50, wherein said unique marker profile further comprises five or more additional pharmaceutically inactive marker substances.

53. The labeled pharmaceutical product of claim 51, wherein at least one of said additional pharmaceutically inactive marker substances is selected from the group consisting of carbohydrates, disaccharides, trisaccharides, tetrasaccharides, oligosaccharides, polysaccharides, isoprenoids, lipids, steroids, acrylic polymers, poloxamers, polyoxyls, polysorbates, acesulfame, an acetylated monoglyceride, butylparaben, povidone, copovidone, crospovidone, gelucire, hypromelloses, polycarbophil, polydextrose, tartaric acid or a salt thereof, and derivatives thereof.

54. The labeled pharmaceutical product of claim 50, wherein said unique marker profile comprises at least one monodisperse polyethylene glycol.

55. The labeled pharmaceutical product of claim 50, wherein said unique marker profile comprises two or more monodisperse polyethylene glycols each having a different molecular weights.

56. The labeled pharmaceutical product of claim 50, wherein unique marker profile encodes the origin of said labeled pharmaceutical product.

57. The labeled pharmaceutical product of claim 50, wherein unique marker profile encodes the intended recipient of said labeled pharmaceutical product.

58. The labeled pharmaceutical product of claim 50, wherein unique marker profile encodes a preselected characteristic of an intended recipient of said labeled pharmaceutical product.
